(19) United States
(12) Patent Application Publication
(10) Pub. No.: US 2019/0183400 A1
(43) Pub. Date: Jun. 20, 2019

(54) COGNITIVE URINARY TRACT TEST AND TREATMENT

(71) Applicant: INTERNATIONAL BUSINESS MACHINES CORPORATION, Armonk, NY (US)

(72) Inventors: Hariklia Deligianni, Alpine, NJ (US); Stephen W. Bedell, Wappingers Falls, NY (US); Bruce B. Doris, Slingerlands, NY (US)

(21) Appl. No.: 15/843,199

(22) Filed: Dec. 15, 2017

(51) Int. Cl. A61B 5/20; H04N 5/225; H04N 5/374; H04N 5/232; G06T 7/00; A61B 1/00; A61B 1/04; A61B 1/307; A61B 5/07; A61B 5/00; A61B 17/22

(52) U.S. Cl. CPC A61B 5/201; G16H 40/63; H04N 5/374; H04N 5/23293; G06T 7/0014; A61B 1/00156; A61B 1/041; A61B 1/307; A61B 5/07; A61B 5/7264; A61B 1/00016; A61B 17/22012; A61B 5/7282; A61B 1/00045; A61B 1/0002; A61B 1/00009; A61B 5/4836; H04N 5/2256

(57) ABSTRACT

A cognitive urinary tract microcomputer is provided. The cognitive urinary tract microcomputer includes a propulsion device for moving the cognitive urinary tract microcomputer through a urinary tract. The cognitive urinary tract microcomputer further includes an image sensor for capturing images of the urinary tract. The cognitive urinary tract microcomputer also includes a computer having an image processor and a memory. The memory stores a cognitive algorithm executed by the image processor for comparing images of normal urinary tract segments to the captured images to detect abnormalities in the urinary tract and provide an indication of the abnormalities to a user.

### BACKGROUND

### Technical Field

The present invention relates generally to medical tests and treatments and, in particular, to a cognitive urinary tract test and treatment.

### Description of the Related Art

Kidney stones in urinary tracts are very painful if not treated early. The stones can grow large and cause additional physical complications such as, for example, infections and so forth.

CAT scans and x-rays can be used to detect kidney stones. However, these methods have drawbacks due to excessive radiation and also if the stone is small the CAT scan or x-ray may not detect the stone.

Conventional stone removal processes involve extracting the stone with a catheter type device which can move the stone back into the kidney and lead to additional complications. Alternatively, an ultrasonic bath can be used to break up the stones but this approach also has drawbacks.

Hence, there is a need for an improved urinary tract test and treatment.

### SUMMARY

According to an aspect of the present invention, a cognitive urinary tract microcomputer is provided. The cognitive urinary tract microcomputer includes a propulsion device for moving the cognitive urinary tract microcomputer through a urinary tract. The cognitive urinary tract microcomputer further includes an image sensor for capturing images of the urinary tract. The cognitive urinary tract microcomputer also includes a computer having an image processor and a memory. The memory stores a cognitive algorithm executed by the image processor for comparing images of normal urinary tract segments to the captured images to detect abnormalities in the urinary tract and provide an indication of the abnormalities to a user.

According to another aspect of the present invention, a method is provided for deploying a cognitive microcomputer in a urinary tract. The method includes moving, by a propulsion device of the cognitive microcomputer, the cognitive urinary tract microcomputer through the urinary tract. The method further includes capturing, by an image sensor of the cognitive microcomputer, images of the urinary tract. The method also includes executing, by a computer of the cognitive microcomputer, a cognitive algorithm for comparing images of normal urinary tract segments to the captured images to detect abnormalities in the urinary tract and provide an indication of the abnormalities to a user. The computer has an image processor and a memory. The memory stores the cognitive algorithm executed by the image processor.

According to yet another aspect of the present invention, a method is provided for forming a cognitive urinary tract microcomputer. The method includes configuring a propulsion device to provide movement through a urinary tract. The method further includes configuring an image sensor to capture images of the urinary tract. The method also includes configuring a computer having an image processor and a memory such that the memory stores a cognitive algorithm executed by the image processor to compare images of normal urinary tract segments to the captured images to detect abnormalities in the urinary tract and provide an indication of the abnormalities to a user. The method additionally includes operatively connecting the propulsion device, the image sensor, and the computer to form the cognitive urinary tract microcomputer as a single encapsulated unit.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 shows an exemplary environment to which the present invention can be applied, in accordance with an embodiment of the present invention;

FIG. 2 shows an exemplary cognitive urinary tract microcomputer, in accordance with an embodiment of the present invention;

FIG. 3 further shows the microcomputer of FIG. 2, in accordance with an embodiment of the present invention;

FIG. 4 further shows the camera/light source/piezoelectric module of FIG. 2, in accordance with an embodiment of the present invention;

FIG. 5 further shows the propulsion device of FIG. 2, in accordance with an embodiment of the present invention;

FIG. 6 shows an exemplary method for using a cognitive urinary tract microcomputer, in accordance with an embodiment of the present invention;

FIG. 7 shows another exemplary method for using a cognitive urinary tract microcomputer, in accordance with an embodiment of the present invention; and

FIG. 8 shows an exemplary method for forming a cognitive urinary tract microcomputer, in accordance with an embodiment of the present invention.

### DETAILED DESCRIPTION

The present invention is directed to a cognitive urinary tract test and treatment.

In an embodiment, the present invention provides a cognitive urinary tract microcomputer capable of analyzing a urinary tract, identifying abnormalities such as kidney stones in the urinary tract, and treating the abnormalities (e.g., kidney stones) locally without external assistance.

In an embodiment, the present invention can capture images in a urinary tract using a camera sensor, perform comparisons of the captured images to normal urinary tract segments to detect abnormalities such as kidney stones in the urinary tract, and selectively apply ultrasound to the kidney stones to dissolve them. The selective application of ultrasound can be based on a cognitive decision made based on the aforementioned comparisons.

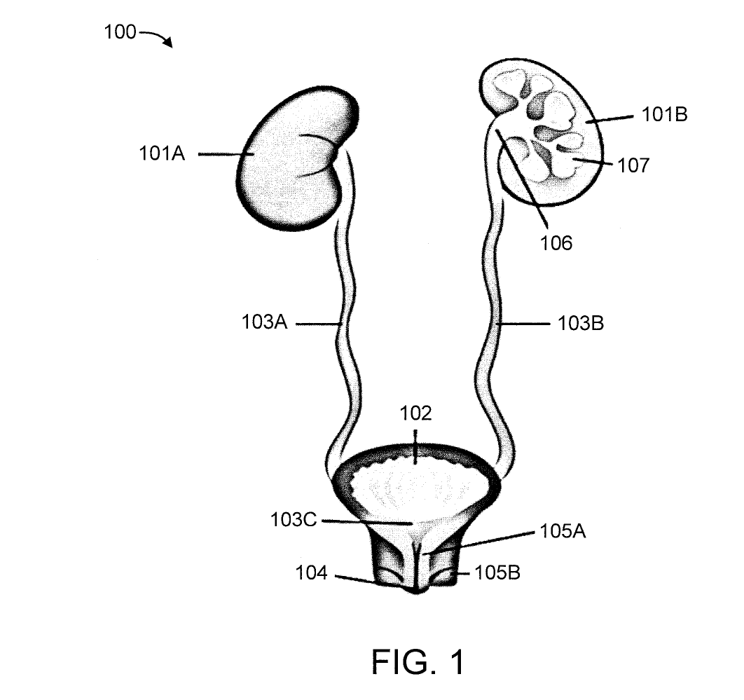

FIG. 1 shows an exemplary environment 100 to which the present invention can be applied, in accordance with an embodiment of the present invention.

The environment 100 involves the urinary tract of the human body. The urinary tract is the body's drainage system for removing urine, which is composed of wastes and extra fluid.

In particular, the environment 100 involves a pair of kidneys 101A and 101B, each connected to a bladder 102 via a pair of ureters 103A and 103B. An inside view of kidney 101B is shown. Also shown are the opening of the ureter (ureteral orifice) 103C, a urethra 104, an internal sphincter 105A, an external sphincter 105B, a renal pelvis 106 and calyx 107.

The kidneys 101A and 101B are two bean-shaped organs that filter about 120 to 150 quarts of blood each day to produce about 1 to 2 quarts of urine. The renal pelvis 106 is a part of a ureter (ureter 103B in the case of FIG. 1) that acts as a funnels for urine flowing to that ureter. The calyx 107 is a branch of the renal pelvis 106.

The ureters 103A and 103B are the thin tubes of muscle, one on each side of the bladder 102, that carry urine from each of the kidneys 101A and 101B to the bladder 102.

The bladder 102, located in the pelvis between the pelvic bones, is a hollow, muscular, balloon-shaped organ that expands as it fills with urine. Bladder 102 emptying is known as urination. During urination, the bladder 102 empties through the urethra 104, located at the bottom of the bladder 102. Hence, the ureters 103A and 103B, bladder 102, and urethra 104 move urine from the kidneys 101A and 101B and store it until releasing it from the body. The internal sphincter 105A and external sphincter 105B control the flow of urine from the body.

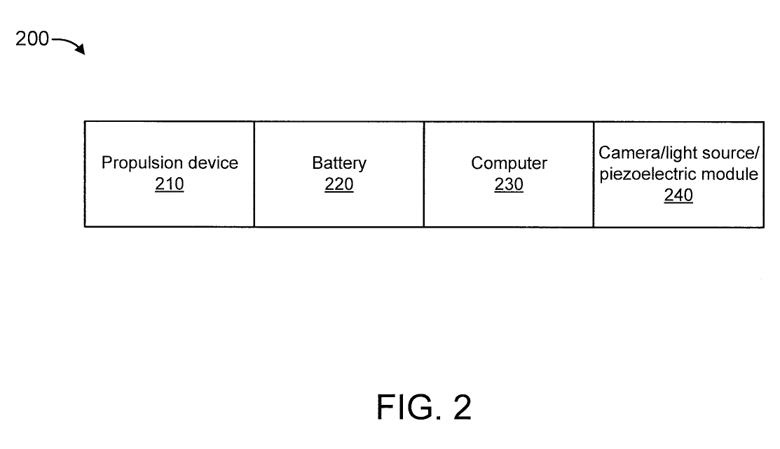

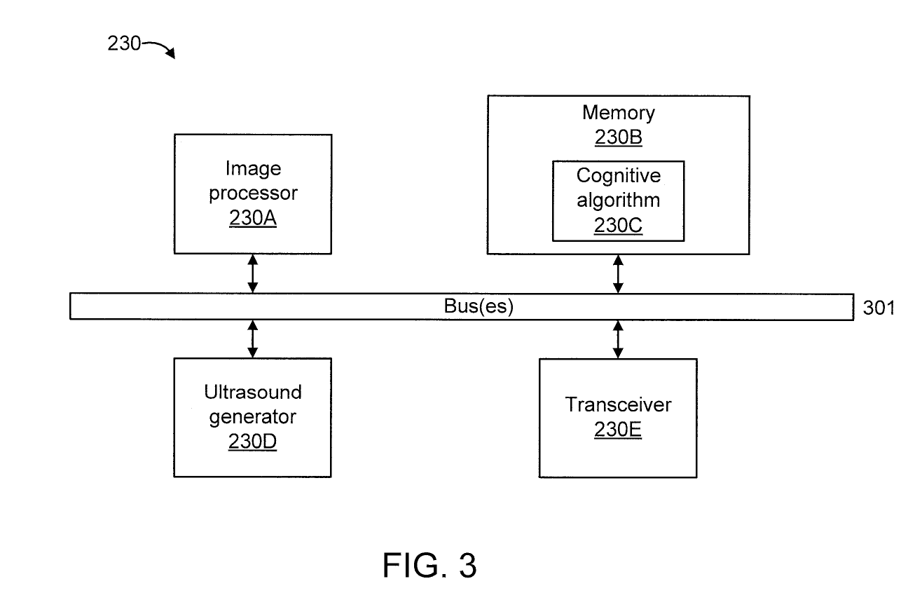

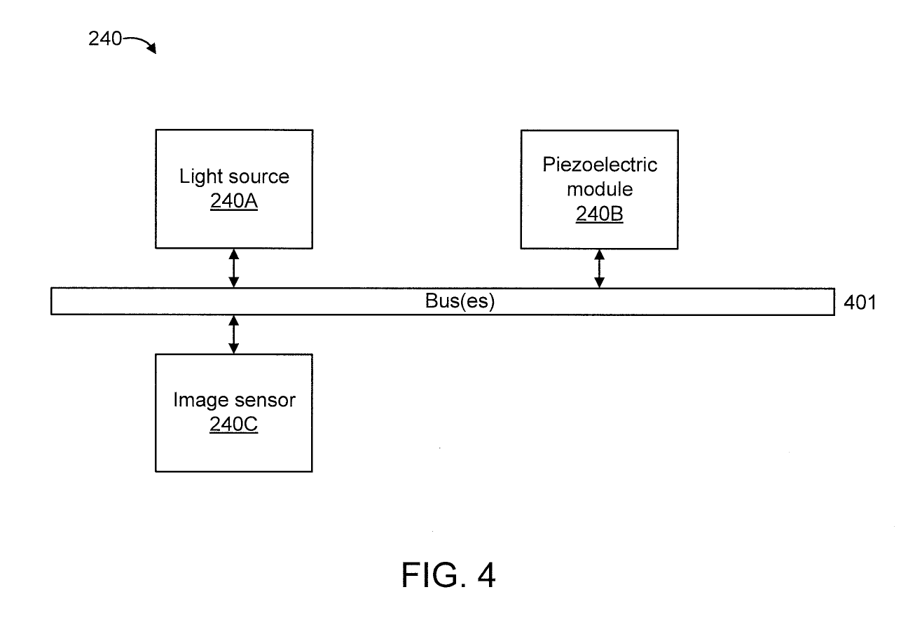

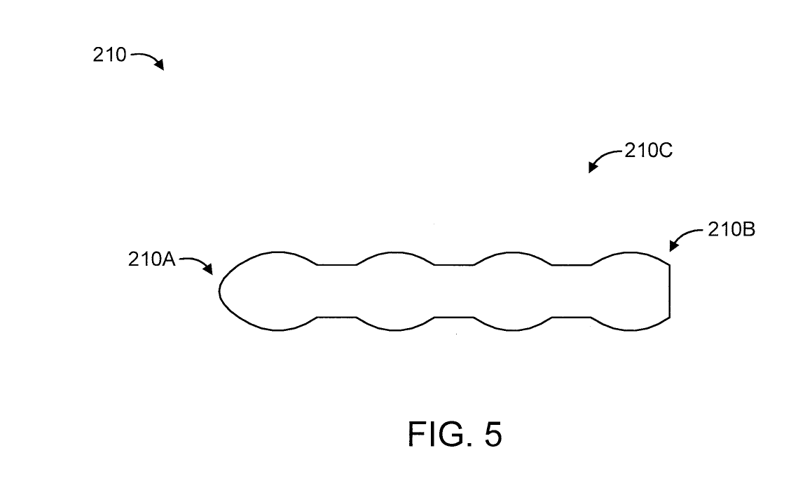

FIG. 2 shows an exemplary cognitive urinary tract microcomputer 200, in accordance with an embodiment of the present invention. FIG. 3 further shows the microcomputer 230 of FIG. 2, in accordance with an embodiment of the present invention. FIG. 4 further shows the camera/light source/piezoelectric module 240 of FIG. 2, in accordance with an embodiment of the present invention. FIG. 5 further shows the propulsion device 210 of FIG. 2, in accordance with an embodiment of the present invention. It is to be appreciated that the cognitive urinary tract microcomputer and constituent elements depicted in FIGS. 2-5 are not drawn to scale, but instead are drawn with exaggerated sizes for the sakes of illustration and clarity. Moreover, the shapes of the element are not necessarily representative of an actual device shape. As can be appreciated by one of ordinary skill in the art, the size and shape of the elements and the overall device can be varied depending upon the implementation. In an actual implementation, the cognitive urinary tract microcomputer is a small device capable of being inserted and operating within the urinary tract. Nonetheless, FIG. 5 shows an exemplary shape of the propulsion unit 210, noting that other shapes can also be used.

The cognitive urinary tract microcomputer 200 can analyze a urinary tract, identify abnormalities such as kidney stones in the urinary tract, and treat the abnormalities (e.g., kidney stones) locally without external assistance.

The cognitive urinary tract microcomputer 200 includes a propulsion device 210, a battery 220, a microcomputer 230, and a camera/light source/piezoelectric module (hereinafter “camera” or “image sensor” in short) 240.

The propulsion device 210 moves the cognitive urinary tract microcomputer 200 through the urinary tract. For example, the propulsion device 210 can include a moving member (e.g., an oscillating member, an articulating member, and so forth) for facilitating movement through the urinary tract. For example, an articulating member 510 such as, for example, a tail-like member or a worm-like member or snake-like member, having an untethered back end 510A and a front end 510B connected to another element (e.g., the computer 230), can be used to facilitate movement through the urinary tract. The articulating member 510 may have multiple segments 510C that facilitate movement. These and other types of propulsion devices are readily determined by one of ordinary skill in the art, given the teachings of the present invention provided herein, while maintaining the spirit of the present invention.

The battery 220 supplies power to one or more elements of the cognitive urinary tract microcomputer 200 such that the cognitive urinary tract microcomputer 200 can act autonomously (e.g., without having to be guided by a catheter after being inserted) within a human body.

The camera 240 captures images of the urinary tract. In an embodiment, the camera includes a Complimentary Metal-Oxide Semiconductor (CMOS) image sensor. Of course, other types of cameras/image sensors can be used in accordance with the teachings of the present invention, while maintaining the spirit of the present invention. The camera 240 can include a light source 240A for illuminating the urinary tract and an image sensor 240B for capturing images of the urinary tract. The camera 240 can also include a piezoelectric module 240B. In an embodiment, the piezoelectric module 240B can be used to supply power to at least the camera 240. In an embodiment, the piezoelectric module 240B can be used to generate ultrasound waves (e.g., using the inverse piezoelectric effect).

The microcomputer 230 can include an image processor 230A, a memory 230B, and a cognitive algorithm 230C (e.g., stored on the memory and executed by the processor 230A) to compare images of normal urinary tract segments to captured images. When an abnormality is observed, the microcomputer 230 can determine if it is a kidney stone. Moreover, in an embodiment, the microcomputer 230 can include an ultrasound generator 230D for selectively generating ultrasound waves for dissolving the kidney stone. In another embodiment, the piezoelectric module 240B of the image sensor 240 can be used to generate ultrasound waves for dissolving the kidney stone.

In an embodiment, the microcomputer 230 can further include a transceiver 230E for transmitting captured images and/or analysis results to an external device (e.g., a display device, an external computer having a display device, and/or so forth). Moreover, the transceiver 230E can be used to provide instructions, updates (software, firmware, etc.), and/or other information to the cognitive urinary tract microcomputer 200.

For the sake of illustration, a particular configuration is shown in FIG. 3 of the elements in the microcomputer 230. Also for the sake of illustration, a particular configuration is shown in FIG. 4 of the elements of the camera/light source/piezoelectric module 240. In the configuration of FIG. 3, the elements are interconnected by one or more buses 301, and in the configuration of FIG. 4, the elements are interconnected by one or more buses 401. However, as readily appreciated by one of ordinary skill in the art, other configurations can also be used, given the teachings of the present invention provided herein, while maintaining the spirit of the present invention.

It is to be appreciated that while the propulsion device 210, the battery 220, the microcomputer 230, and the camera 240 are shown as separate elements, in other embodiments, one or more of these elements can be combined into a single element. Similarly, while the microcomputer 230 is described as including an image processor 230A, a memory 230B, a cognitive algorithm 230C, an ultrasound generator 230D, and a transceiver 230E, one or more of these elements can be made separate from the others. These and other variations and configurations of the elements of cognitive urinary tract microcomputer 200 are readily determined by one of ordinary skill in the art, given the teachings of the present invention provided herein, while maintaining the spirit of the present invention.

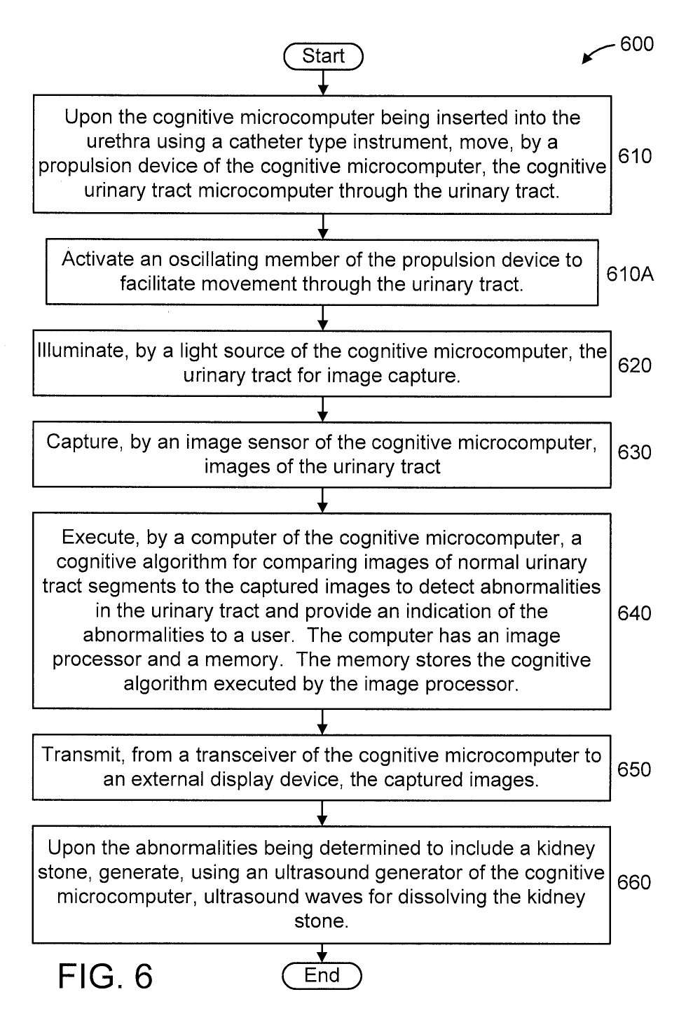

FIG. 6 shows an exemplary method 600 for using a cognitive urinary tract microcomputer, in accordance with an embodiment of the present invention.

At step 610, upon the cognitive microcomputer being inserted into the urethra using a catheter type instrument, move, by a propulsion device of the cognitive microcomputer, the cognitive urinary tract microcomputer through the urinary tract.

In an embodiment, step 610 can include step 610A.

At step 610A, activate an oscillating member of the propulsion device to facilitate movement through the urinary tract.

At step 620, illuminate, by a light source of the cognitive microcomputer, the urinary tract for image capture.

At step 630, capture, by an image sensor of the cognitive microcomputer, images of the urinary tract.

At step 640, execute, by a computer of the cognitive microcomputer, a cognitive algorithm for comparing images of normal urinary tract segments to the captured images to detect abnormalities in the urinary tract and provide an indication of the abnormalities to a user. The computer has an image processor and a memory. The memory stores the cognitive algorithm executed by the image processor.

At step 650, transmit, from a transceiver of the cognitive microcomputer to an external display device, the captured images.

At step 660, upon the abnormalities being determined to include a kidney stone, generate, using an ultrasound generator of the cognitive microcomputer, ultrasound waves for dissolving the kidney stone.

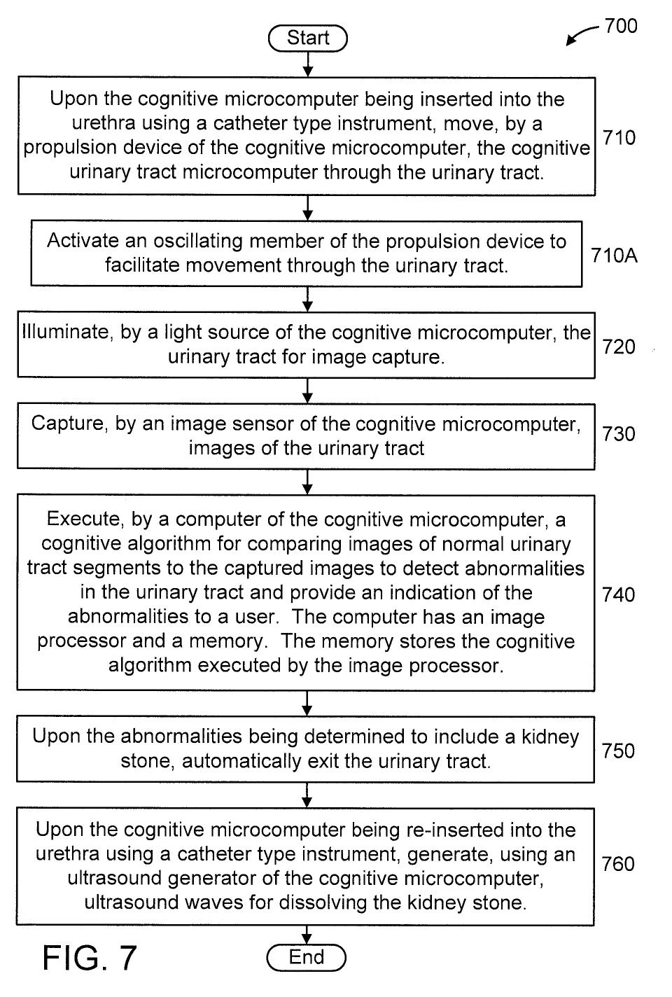

FIG. 7 shows another exemplary method 700 for using a cognitive urinary tract microcomputer, in accordance with an embodiment of the present invention.

At step 710, upon the cognitive microcomputer being inserted into the urethra using a catheter type instrument, move, by a propulsion device of the cognitive microcomputer, the cognitive urinary tract microcomputer through the urinary tract.

In an embodiment, step 710 can include step 710A.

At step 710A, activate an oscillating member of the propulsion device to facilitate movement through the urinary tract.

At step 720, illuminate, by a light source of the cognitive microcomputer, the urinary tract for image capture.

At step 730, capture, by an image sensor of the cognitive microcomputer, images of the urinary tract.

At step 740, execute, by a computer of the cognitive microcomputer, a cognitive algorithm for comparing images of normal urinary tract segments to the captured images to detect abnormalities in the urinary tract and provide an indication of the abnormalities to a user. The computer has an image processor and a memory. The memory stores the cognitive algorithm executed by the image processor.

At step 750, upon the abnormalities being determined to include a kidney stone, automatically exit the urinary tract. The exit can be made to, for example, permit access to the captured images, further consider the proper course of action, collect additional data, and so forth.

At step 760, upon the cognitive microcomputer being re-inserted into the urethra using a catheter type instrument, generate, using an ultrasound generator of the cognitive microcomputer, ultrasound waves for dissolving the kidney stone.

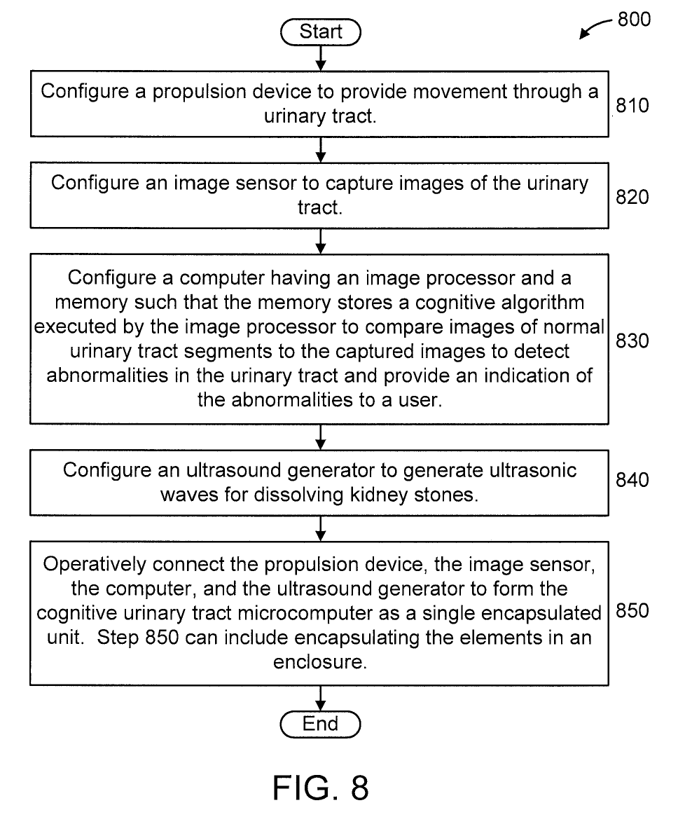

FIG. 8 shows an exemplary method 800 for forming a cognitive urinary tract microcomputer, in accordance with an embodiment of the present invention.

At step 810, configure a propulsion device to provide movement through a urinary tract.

At step 820, configure an image sensor to capture images of the urinary tract. The image sensor can include a light source that is configured to illuminate the urinary tract for image capture.

At step 830, configure a computer having an image processor and a memory such that the memory stores a cognitive algorithm executed by the image processor to compare images of normal urinary tract segments to the captured images to detect abnormalities in the urinary tract and provide an indication of the abnormalities to a user.

At step 840, configure an ultrasound generator to generate ultrasonic waves for dissolving kidney stones.

At step 850, operatively connect the propulsion device, the image sensor, the computer, and the ultrasound generator to form the cognitive urinary tract microcomputer as a single encapsulated unit. Step 850 can include encapsulating the elements in an enclosure. The enclosure can be made from Titanium and/or some other material(s) that is preferably hypoallergenic and suitable for insertion into a body.

In an embodiment, one or more of the elements forming the cognitive urinary tract microcomputer can be formed together, for example, using one or more integrated circuits or a single or set of chips and so forth. For example, one or more elements can be implemented by a variety of devices, which include but are not limited to, Digital Signal Processing (DSP) circuits, programmable processors, Application Specific Integrated Circuits (ASICs), Field Programmable Gate Arrays (FPGAs), Complex Programmable Logic Devices (CPLDs), and so forth. These and other variations of the elements used to form a cognitive urinary tract microcomputer in accordance with the present invention are readily determined by one of ordinary skill in the art, given the teachings of the present invention provided herein, while maintaining the spirit of the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A cognitive urinary tract microcomputer, comprising:
a propulsion device for moving the cognitive urinary tract microcomputer through a urinary tract;
an image sensor for capturing images of the urinary tract; and
a computer having an image processor and a memory, the memory storing a cognitive algorithm executed by the image processor for comparing images of normal urinary tract segments to the captured images to detect abnormalities in the urinary tract and provide an indication of the abnormalities to a user.

2. The cognitive urinary tract microcomputer of claim 1, wherein the abnormalities comprise a kidney stone, and the cognitive urinary tract microcomputer further comprises an ultrasound generator for generating ultrasound waves for dissolving the kidney stone.

3. The cognitive urinary tract microcomputer of claim 1, wherein the ultrasound generator comprises a piezoelectric module for generating the ultrasound waves.

4. The cognitive urinary tract microcomputer of claim 1, further comprising a battery for powering the cognitive microcomputer such that the cognitive microcomputer can act autonomously within the urinary tract.

5. The cognitive urinary tract microcomputer of claim 1, wherein the image sensor comprises a Complimentary Metal-Oxide Semiconductor image sensor.

6. The cognitive urinary tract microcomputer of claim 1, wherein the propulsion device comprises an oscillating member for facilitating movement through the urinary tract.

7. The cognitive urinary tract microcomputer of claim 1, wherein the computer is configured to automatically exit the urinary tract upon detecting the abnormality to permit access to the captured images.

8. The cognitive urinary tract microcomputer of claim 1, further comprising a transceiver for transmitting the captured images to an external display device.

9. The cognitive urinary tract microcomputer of claim 1, wherein the image sensor comprises a light source for illuminating the urinary tract for image capture.

10. The cognitive urinary tract microcomputer of claim 1, wherein the image sensor comprises a piezoelectric module for generating ultrasound waves for kidney stone dissolving.

11. A method for deploying a cognitive microcomputer in a urinary tract, comprising:
moving, by a propulsion device of the cognitive microcomputer, the cognitive urinary tract microcomputer through the urinary tract;
capturing, by an image sensor of the cognitive microcomputer, images of the urinary tract; and
executing, by a computer of the cognitive microcomputer, a cognitive algorithm for comparing images of normal urinary tract segments to the captured images to detect abnormalities in the urinary tract and provide an indication of the abnormalities to a user, the computer having an image processor and a memory, the memory storing the cognitive algorithm executed by the image processor.

12. The method of claim 11, wherein the abnormalities comprise a kidney stone, and the method further comprises generating, using an ultrasound generator of the cognitive microcomputer, ultrasound waves for dissolving the kidney stone.

13. The method of claim 11, wherein the abnormalities comprise a kidney stone, and the method further comprises generating, using a piezoelectric module of the cognitive microcomputer, ultrasound waves for dissolving the kidney stone.

14. The method of claim 11, further comprising powering, by a battery, the cognitive microcomputer such that the cognitive microcomputer can act autonomously within the urinary tract.

15. The method of claim 11, wherein said moving step comprises activating an oscillating member of the propulsion device to facilitate movement through the urinary tract.

16. The method of claim 11, further comprising automatically exiting the urinary tract upon detecting the abnormality to permit access to the captured images.

17. The method of claim 11, further comprising receiving, from a transceiver of the cognitive microcomputer by an external display device, the captured images.

18. The method of claim 11, further comprising illuminating, by a light source of the image sensor, the urinary tract for image capture.

19. A method for forming a cognitive urinary tract microcomputer, the method comprising:
configuring a propulsion device to provide movement through a urinary tract;
configuring an image sensor to capture images of the urinary tract;
configuring a computer having an image processor and a memory such that the memory stores a cognitive algorithm executed by the image processor to compare images of normal urinary tract segments to the captured images to detect abnormalities in the urinary tract and provide an indication of the abnormalities to a user; and
operatively connecting the propulsion device, the image sensor, and the computer to form the cognitive urinary tract microcomputer as a single encapsulated unit.

20. The method of claim 19, wherein the method further comprises:
configuring an ultrasound generator to generate ultrasonic waves for dissolving kidney stones; and
operatively coupling the ultrasound generator to the cognitive urinary tract microcomputer.
